(19) United States
(12) Patent Application Publication
(10) Pub. No.: US 2023/0381509 A1
(43) Pub. Date: Nov. 30, 2023

(54) Use of Non-Invasive Sensory Systems to Titrate Cranial Nerve Stimulation to Enhance Brain Clearance Closed-Loop

(71) Applicant: Wisconsin Alumni Research Foundation, Madison, WI (US)

(72) Inventors: Kip Ludwig, Middleton, WI (US); Nishant Verma, Madison, WI (US); Justin Williams, Cambridge, WI (US)

(21) Appl. No.: 18/201,887

(22) Filed: May 25, 2023

Related U.S. Application Data
(60) Provisional application No. 63/346,040, filed on May 26, 2022.

(51) Int. Cl. A61N 1/36

(52) U.S. Cl. CPC A61N 1/36025; A61N 1/36031; A61N 1/36034

(57) ABSTRACT

Real-time sensing of cerebrospinal fluid (CSF) clearance can be used to optimize electrode target engagement of the peripheral cranial nerves and detect associated changes within the connected nerve truck and brain. This CSF clearance data may be used in “open loop” systems to inform operator-controlled programming or sent directly to the stimulator itself to titrate programming “closed loop” on the device.

### CROSS-REFERENCE TO RELATED APPLICATIONS

This application claims the benefit of U.S. Provisional Patent Application No. 63/346,040, filed May 26, 2022, which is incorporated by reference herein in its entirety.

### STATEMENT REGARDING FEDERALLY SPONSORED RESEARCH OR DEVELOPMENT

This invention was made with government support under N66001-17-2-4010 awarded by the DOD/DARPA. The government has certain rights in the invention.

### BACKGROUND OF THE INVENTION

The present invention relates to electrical stimulation of target nerves to enhance waste clearance in the brain, and specifically, to sensing systems for optimizing target activation and titrating nerve stimulation protocols in real-time.

Waste removal from the central nervous system (CNS) is essential for maintaining brain homeostasis. Disruption of waste clearance can lead to protein accumulation. The aggregation of pathogenic proteins β-amyloid, α-synuclein, and C-tau in the brain may cause the deleterious effects of numerous diseases and disorders such as traumatic brain injury/chronic traumatic encephalopathy, epilepsy, Alzheimer's disease, and Parkinson's disease. Removal of these pathogenic proteins has been found to have substantial therapeutic benefit, for example, in treating traumatic brain injury/chronic traumatic encephalopathy, epilepsy, Alzheimer's disease, and Parkinson's disease. Disruption of glymphatic waste clearance is also implicated in several mental health disorders including depression, bipolar disorder (BPD), and anxiety.

The glymphatic system, or glymphatic clearance pathway, is a macroscopic waste clearance system for the vertebrate CNS utilizing a unique system of perivascular tunnels formed by glial cells to promote efficient elimination of soluble and insoluble proteins and metabolites from the CNS.

Specifically, recent studies have shown that cerebrospinal fluid (CSF) and interstitial fluid (ISF) continuously interchange to clear pathogenic proteins and metabolic byproducts from the brain. This exchange is facilitated by convective influx of CSF along the paravascular spaces. From the subarachnoid space, CSF is driven into the Virchow-Robin spaces by a combination of arterial wall movement, respiration, and CSF pressure gradients. The subsequent transport of CSF into the brain parenchyma is mediated by aquaporin-4 (AQP4) water channels. The movement of CSF through the brain parenchyma further drives convective ISF fluxes within the tissue toward the perivenous spaces from where it drains out of the brain. Therefore, the glymphatic system has been found to play an important role in clearing pathogenic proteins and metabolic byproducts from the brain, separate from the BBB.

Increasing the penetration of CSF into the brain parenchyma can serve many therapeutic purposes, including diluting endogenous neurochemical transmitter concentrations within the brain, altering the clearance rates of drugs delivered orally that penetrate through the blood brain barrier or delivered via a catheter system to the brain, and reducing non-synaptic coupling between neurons to treat diverse conditions including anxiety disorders, tremor, and seizure.

### SUMMARY OF THE INVENTION

The present inventors have found that 1) inducing arterial wall movement, 2) inducing AQP4 channel polarization and/or increasing gaps in the astrocytic end-feet, and 3) reducing neural activity to increase fluid in the extracellular space reducing resistance to fluid movement are factors that play a role in CSF/ISF exchange. Wall movement refers to vessel wall dynamics such as pulsatility, vasodilation, and vasoconstriction which occur in the paravascular space and may contribute to pushing CSF along the paravascular space and into the parenchyma.

The present inventors have also found that real-time sensing of CSF clearance, and/or its secondary biomarkers, can be used to optimize electrode target engagement of the peripheral cranial nerves and detect associated changes within the connected nerve truck and brain. This data can be used in “open loop” systems to inform operator-controlled programming or can be sent directly to the electrical stimulator itself to titrate programming “closed loop” on the stimulation device.

In one embodiment of the present invention, the “closed loop” systems may include the use of non-invasive electroencephalogram (EEG) sensors to detect activation within targeted brain regions (e.g., hippocampus/prefrontal cortex) as well as to detect the frequency power of induced oscillations within the brain (e.g., 2 Hz and 40 Hz targets), data which may be used as inputs into the cranial nerve stimulator to optimize stimulation parameters.

In an alternative embodiment of the present invention, non-invasive and minimally invasive microneurography can be used at target peripheral nerves to ensure activation of the superficial sensory receptors and connected nerve endings to create the desired activation within the nerve trunk projecting to the brain, as well as to confirm that the fiber types necessary for therapeutic activation are being engaged.

In one embodiment of the present invention, an electrical stimulation device is provided for stimulating cranial nerves to improve waste clearance through a glymphatic system or meningeal lymphatic system of a human patient. The electrical stimulation device includes a nerve stimulator configured to stimulate a cranial nerve of the human patient. A nerve stimulator is configured to generate at least one carrier wave having a first carrier amplitude and a first carrier frequency. A modulator receives the at least one carrier wave and a modulation wave to modulate the at least one carrier wave for application to the at least one electrode. An electrical modulation generator generates the modulation wave having a predetermined periodicity providing a first period of stimulation and a second period of relaxation of no stimulation, the predetermined periodicity selected to increase wall movement over continuous stimulation of the glymphatic system or meningeal system by the carrier frequency. A sensing device measures cerebral spinal fluid (CSF)/interstitial fluid (ISF) flow in the brain of the human patient. A controller is configured to adjust at least one of the at least one carrier wave and the modulation wave to increase cerebral spinal fluid (CSF)/interstitial fluid (ISF) flow in response to a measurement of the sensing device.

It is thus a feature of at least one embodiment of the present invention to inform electrical stimulation protocols of the stimulator in real-time using target nerve engagement and brain activity sensing modalities during electrical stimulation.

The controller may be configured to adjust the predetermined periodicity of the modulation wave.

It is thus a feature of at least one embodiment of the present invention to maximize arterial wall movement by interweaving high frequency stimulation with periods of rest to allow the arterial tissue to recovery by monitoring CSF clearance or indicators in real-time.

The controller may be configured to adjust the first carrier frequency.

It is thus a feature of at least one embodiment of the present invention to promote AQP4 mediated CSF/ISF exchange (consistent with slow-wave sleep) by monitoring CSF clearance or indicators of CSF clearance in real-time.

The controller may be configured to adjust the first carrier amplitude.

It is thus a feature of at least one embodiment of the present invention to maximize cortical plasticity in the brain which exhibits optimal effects compared to lower and higher amplitudes by monitoring CSF clearance or indicators of CSF clearance in real-time.

The measurement of the sensing device may be a measure of an electrical activity of the brain. The sensing device may be an electroencephalogram (EEG) machine or magnetoencephalography (MEG) machine.

It is thus a feature of at least one embodiment of the present invention to detect electrical activation within the connected nerve trunk and brain regions of the hippocampus and prefrontal cortex and further detect frequency power of induced oscillations in the brain using noninvasive techniques.

The measurement of the sensing device may be a measure of blood perfusion in the brain. The sensing device may be at least one of a computerized topography (CT) scanner, magnetic resonance imaging (MM) scanner, functional magnetic resonance imaging (fMRI) scanner, positron emission tomography (PET) scanner, transcranial ultrasound, and single-photon emission computed tomography (SPECT) scanner.

It is thus a feature of at least one embodiment of the present invention to detect blood flow changes indicative of CSF clearance within the brain using non-invasive neuroimaging.

The sensing device may be a pupilometer, and the measurement is a dilation of the pupil. The sensing device may be a functional near-infrared spectroscopy (fNIRS) measuring changes in hemoglobin in cerebral blood. The sensing device may measure blood perfusion in the skin of the face or head. The sensing device may measure an evoked neural signal in the facial or trigeminal nerves.

It is thus a feature of at least one embodiment of the present invention to detect electrical activity in the brain using sensitive biological measurements that can track stimulation quickly and in real-time.

The at least one electrode may be adapted to stimulate at least one of a trigeminal nerve, buccal branch nerve, mental branch nerve, facial branch nerve, vagus branch nerve (e.g., auricular vagus nerve), cervical nerve, sympathetic trunk/sympathetic ganglia, and sympathetic efferent branches.

It is thus a feature of at least one embodiment of the present invention to use target nerve engagement that has direct sympathetic/parasympathetic innervation of the cerebral vasculature increasing the speed of dilation and constriction after stimulation.

The at least one carrier wave may include first and second carrier waves of first and second different frequencies and which are delivered simultaneously to the at least one electrode.

It is thus a feature of at least one embodiment of the present invention to provide synergistic effects to stimulation protocols promoting AQP4 mediated CSF/ISF exchange and increasing wall movement, and optimizing protocols based on the effects of both delivered simultaneously under preferred protocols.

The device may further include sensors detecting salivary biomarkers indicating a change to CSF flow selected from at least one of amyloid beta peptide, tau protein, lactoferrin, alpha-synuclein, DJ-1 protein, chromogranin A, huntingtin protein, DNA methylation disruptions, and micro-RNA.

It is thus a feature of at least one embodiment of the present invention to include biological sensors directly on, for example, an intraoral stimulation device that can provide additional indications of increased CSF flow related to biological markers.

An alternative embodiment of the invention provides a method of modifying waste clearance through a glymphatic system or meningeal lymphatic system of a patient including: positioning at least one electrode in close proximity to a nerve of the patient; generating a carrier wave having a carrier frequency; generating a modulation wave having a predetermined periodicity providing a first period of stimulation and a second period of no stimulation, the predetermined periodicity selected to increase waste clearance over continuous stimulation of the glymphatic system or meningeal system by the first carrier wave; modulating the carrier wave and applying the carrier wave to the electrode; measuring a cerebral spinal fluid (CSF)/interstitial fluid (ISF) flow in the brain of the patient; and adjusting the carrier frequency and the modulation wave to increase CSF/ISF flow in response to a measurement of the cerebral spinal fluid (CSF)/interstitial fluid (ISF) flow in the brain of the patient.

It is thus a feature of at least one embodiment of the present invention to use real-time sensing protocol (1) to optimize AQP4 mediated fluid exchange, (2) the use of temporal patterning and stimulation parameters to maximize the cerebral wall movement, (3) promotes phagocytic phenotype in glial cells to break down waste proteins and (4) reduces neural activity to decrease fluid within the neuron, thereby increasing fluid in the extracellular space to reduce the resistance of the parenchymal pathway to fluid flow promoting clearance, to synergistically increase the CSF flow rate to the periarterial spaces to maximize waste clearance from the brain. The wall movement may be defined as a change in the vessel diameter over time relative to a mean vessel diameter.

The method may further comprise measuring an electrical activity of the brain. The method may further comprise measuring a blood perfusion in the brain.

It is thus a feature of at least one embodiment of the present invention to approximate cerebral spinal fluid (CSF)/interstitial fluid (ISF) flow levels and clearance using known brain activity measurement modalities.

The method may further comprise adjusting a position of the at least one electrode in response to the measurement of the cerebral spinal fluid (CSF)/interstitial fluid (ISF) flow in the brain of the patient.

It is thus a feature of at least one embodiment of the present invention to correct poor placement of stimulating electrodes prior to electrical stimulation treatment.

The method may further comprise adjusting a delivery time of the at least one carrier frequency in response to the measurement of the cerebral spinal fluid (CSF)/interstitial fluid (ISF) flow in the brain of the patient and according to a measure of electrical activity of the brain.

It is thus a feature of at least one embodiment of the present invention to deliver minimal amounts of electrical stimulation treatment necessary to elicit desired response.

A second carrier wave may have a second carrier frequency wherein the second carrier frequency is less than the first carrier frequency and delivered simultaneously with the first carrier wave. In some embodiments, a second carrier wave may have a second carrier frequency wherein the second carrier frequency is greater than the first carrier frequency and delivered simultaneously with the first carrier wave.

The method may further comprise adjusting the second carrier frequency to increase cerebral spinal fluid (CSF)/interstitial fluid (ISF) flow in response to the measurement of the cerebral spinal fluid (CSF)/interstitial fluid (ISF) flow in the brain of the patient.

The at least one electrode may be positioned over at least one of an inferior alveolar nerve and a mental branch nerve.

These particular objects and advantages may apply to only some embodiments falling within the claims and thus do not define the scope of the invention.

### BRIEF DESCRIPTION OF THE DRAWINGS

FIG. 1 is a schematic diagram of a human skull receiving electrical stimulation from electrodes positioned on facial and lingual nerves of the head in the tooth and jaw region and in the cheek region in accordance with the present invention;

FIG. 2 is a schematic diagram of a human head receiving electrodes on or in a cheek region to deliver directed electrical stimulation to a buccal branch of the lingual nerve within the cheek region and receiving electrodes on a forehead region to deliver directed electrical stimulation to supraorbital branches within the forehead region;

FIG. 3 is a block diagram showing an electrical stimulator delivering at least one carrier wave to a modulator selectively modulating the at least one carrier wave to produce a modified electrical signal providing increased CSF flow through arterial vessels to the head and therefore increasing waste clearance;

FIG. 4 is a graph showing a magnitude of vasodilation/constriction of arterial vessels relative to a mean vessel diameter as a function of a time to return to baseline (TBL) at a given electrical stimulation frequency and duty cycle;

FIG. 5 is a timeline showing the electrical stimulation protocol for delivering layered low frequency modulated and low frequency unmodulated carrier waves during sleep and delivering the layered low frequency carrier waves with a high frequency unmodulated carrier wave in an alternating pattern during wakefulness;

FIG. 6 is a block diagram showing a closed loop control implemented by the electrical stimulator and sensing systems of the present invention; and

FIG. 7 is a block diagram showing an open loop control implemented by the electrical stimulator and sensing systems of the present invention.

### DETAILED DESCRIPTION OF THE INVENTION

### Electrical Stimulation of Target Nerve Locations

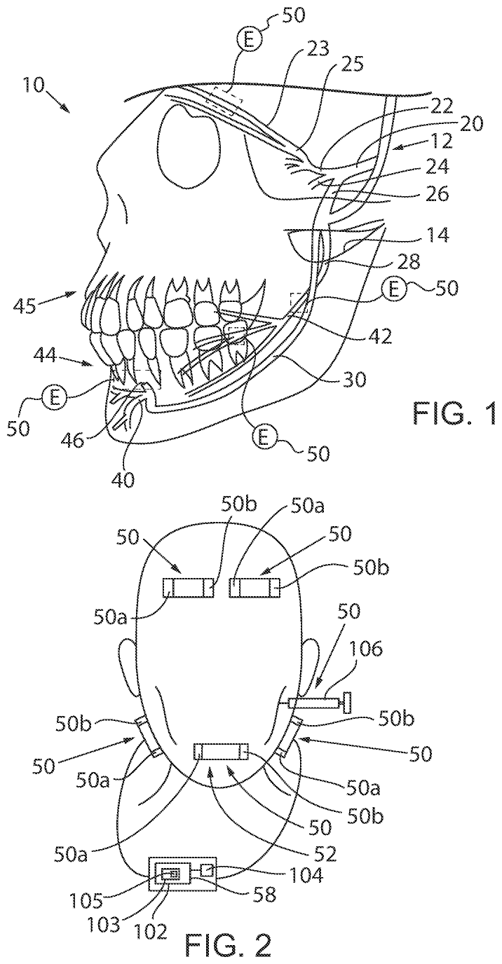

Referring to FIGS. 1 and 2, electrical stimulation of easily accessible neural inputs located outside of the brain and minimally invasive or non-invasive stimulation strategies can induce cardiovascular and respiratory changes, dilate arterial vessels, and increase the wall movement (change in the vessel diameters over time relative to a mean vessel diameter) of penetrating arterial vessels in the brain thus leading to increased clearance of misfolded proteins from the brain. Specifically, electrical stimulation of cranial nerves or local areas around the cranial nerves may selectively cause oscillations in pressure and dilation of arteries that help to improve waste clearance in the brain.

A typical human skull 10 supports a number of cranial nerves 12 emerging directly from the brain, located within the skull 10, and emerging out through cranial foramina 14, or holes, in the skull 10 to reach their final destinations on the exterior of the skull 10 and around the jaw and neck region. These cranial nerves 12 relay information between the brain and other parts of the body.

The trigeminal nerve 20 (fifth cranial nerve) is the largest of the cranial nerves 12 and provides sensation to the face and various motor functions such as biting and chewing functions. The trigeminal nerve 20 includes three major branches: the ophthalmic nerve (V1) 22, the maxillary nerve (V2) 24, and the mandibular nerve (V3) 26. The facial nerve (seventh nerve) has also been shown to be receptive to electrical stimulation.

The mandibular nerve (V3) 26 includes several sub-branches including the lingual nerve 28 and the inferior alveolar nerve 30 that have shown to be particularly receptive to electrical stimulation. The ophthalmic nerve (V1) 22 includes several sub-branches including the supraorbital nerves 23 that has shown to be particularly receptive to electrical stimulation.

Specifically, in a first embodiment, the mental branch nerves 40 are a sub-branch of the inferior alveolar nerve 30 and provides sensation to the front of the chin, lower lip, labial gingiva of the mandibular anterior teeth and the premolars. The buccal branch nerves of trigeminal nerves 42 are a sub-branch of the lingual nerve 28 that provides sensation to the cheek and the second and third molar teeth. The locations of the mental branch nerves 40 and buccal branch nerves of trigeminal nerves 42 in and around the lower jawbone 44 place the stimulation points or respective nerve endings 46 close to the outermost epidermis of the skin making it an ideal location for electrical stimulation where the distance and impedance between an electrode and the target nerve may be minimized. In this respect lower amounts of electrical energy may be needed to stimulate these sensory nerves compared to nerves located deeper within the skin and which may require more invasive procedures to stimulate the nerves.

Electrodes 50 placed at or proximate to the mental branch nerves 40 and buccal branch nerves of trigeminal nerves 42 allow for electrical stimulation of the respective nerves to therefore elicit a sustained response of the arterial vessels to dilate/constrict in a pulsating manner, as further discussed below. In one embodiment of the present invention, the electrodes are positioned over the mental foramen which may transmit electrical stimulation to the terminal branches of the inferior alveolar nerve and vessels of the mental artery.

Alternatively, in a second embodiment, the supraorbital nerves 23 are a sub-branch of the frontal nerve 25 which is a sub-branch of the ophthalmic nerve (V1) 22. The supraorbital nerves 23 provides sensation to the lateral forehead and the upper eyelid. The location of the supraorbital nerves 23 on the forehead places the stimulation points or respective nerve endings 46 close to the outermost epidermis of the skin making it an ideal location for electrical stimulation where the distance and impedance between an electrode 50 and the target nerve may be minimized. In this respect lower amounts of electrical energy may be needed to stimulate these sensory nerves compared to nerves located deeper within the skin and which may require more invasive procedures to stimulate the nerves.

Electrodes 50 placed at or proximate to the supraorbital nerves 23 allow for electrical stimulation of the nerves to therefore elicit a sustained response of the arterial vessels to dilate/constrict in a pulsating manner, as further discussed below. In one embodiment of the present invention, the electrodes 50 are positioned on the forehead which may transmit electrical stimulation to the terminal branches of the supraorbital nerves 23 and vessels of the frontal nerve 25.

Electrical stimulation of specific cranial nerves such as the branches of the trigeminal nerve, i.e., buccal branch nerves, lingual branch nerves, and supraorbital branch nerves, the facial nerve, vagus branch nerves, e.g., auricular vagus nerve, cervical nerve, sympathetic trunk/sympathetic ganglia, and sympathetic efferent branches create a more sustained pulsatile activity compared to stimulation of other cranial nerves. One possible explanation is that the facial and trigeminal nerves have direct sympathetic/parasympathetic innervation of the cerebral vasculature through several routes, including through the sphenopalatine ganglion (SPG) and greater superficial petrosal nerve that innervates the SPG, which are part of neural pathways that directly control the vasodilation/constriction of the cerebral arteries. As a result, the time course for dilation and constriction after a stimulation burst can be quicker than other cranial nerves because the response is quicker than inputs from the spinal cord which change peripheral sympathetic tone or peripheral inputs such as the sciatic nerve that change blood flow primarily through sensory activity mediated neurovascular coupling. Also, stimulation through pathways that change sympathetic/parasympathetic tone outside the brain dilate the peripheral vasculature outside of the brain. The change in blood flow in the brain is primarily in response to this change in peripheral blood flow to maintain perfusion (there are also occasionally indirect connections between the vagus and facial nerve in some subjects). As vagus nerve stimulation only indirectly influences blood flow in cerebral vasculature, it has a slower time constant between burst of stimulation for changes in flow to return to normal.

Electrical stimulation of specific nerve types may be desirable to elicit more CSF response but minimizes pain to the patient. For example, A-delta fibers with small diameter, i.e., 1 to 5 μm, serve to receive and transmit information primarily relating to acute pain (sharp, immediate, and relatively short-lasting) while A-beta fibers with medium diameter, i.e. 6 to 12 μm, and are afferent fibers associated with touch not pain. Therefore, stimulation of group A-beta nerve fibers may be preferred over group A-delta nerve fibers since they are less painful to innervate but provide similar CSF response outcomes.

### Electrical Stimulation Modalities

Referring still to FIGS. 1 and 2, in one embodiment of the present invention, the electrodes 50 may be a part of an intraoral device 52 placed in the mouth, such as a mouthguard, dental filling or dental implant, to electrically stimulate the inferior alveolar nerve 30 and mental branch nerves 40 located near or around the lower jawbone 44 region. The intraoral device 52 may be used to conveniently and consistently position the electrodes 50 at specific locations in the mouth to provide electrical stimulation 72 to target nerves. For example, the electrodes 50 may be placed in close proximity to the mental foramen and/or the inferior alveolar nerve 30 and mental branch nerves 40.

In one embodiment of the present invention, the intraoral device 52 is a mouthpiece receivable into a patient's mouth and configured to carry the electrical circuitry of an electrical stimulator 58, and generally including a processor 102 and battery power supply 104 (for example, including rechargeable lead acid or lithium ion batteries) for delivery of electrical stimulation 72 to the electrodes 50 within the mouth. It is understood that the electrical stimulator 58 may be positioned outside of the mouth and communicate remotely with a controller and electrodes 50 of the intraoral device 52.

The battery power supply 104 may provide power to an electrical stimulator 58 in a manner which allows for current delivery to the electrodes 50. It is understood that an external power supply may also be used to deliver power to the electrical stimulator 58 in addition to or instead of the battery power supply 104.

A desired position of the electrodes 50 on the walls of the intraoral device 52 may be determined by a location of the mental branch nerves 40 and nerve endings 46 in the mouth. In this respect, medical imaging may be used to help locate the position of the mental branch nerves in the mouth and to aid in placement of the electrodes 50 on the intraoral device 52.

The electrodes 50 may include a stimulating cathode electrode 50a placed close to the desired stimulation site, for example, near the nerve endings 46 of the mental branch nerves 40. An anode electrode 50b may then be placed proximal to the cathode electrode 50a with respect to the nerve endings 46. In this respect, the electrical current flows from the anode electrode 50b to the cathode electrode 50a so that the nerve endings 46 receives the stimulus and propagates an action potential upstream through the mental branch nerves 40. The cathode electrode 50a and anode electrodes 50b are spaced apart along an axis generally parallel to the course of the mental branch nerves 40.

Medical imaging or nerve engagement sensing techniques may be used to facilitate precise placement of the cathode electrode 50a close to the nerve ending and the precise placement of the anode electrode 50b upstream from the cathode electrode 50a and proximal to the nerve ending 46, as discussed in further detail below. The cathode electrode 50a may be placed less than 2 cm or less than 1 cm from the mental branch nerve ending 46 and the anode electrode 50b may be placed less than 3 cm or less than 2 cm upstream from the cathode electrode 50a along the mental branch nerves 40.

It is understood that the pair of communicating electrodes, i.e., the cathode electrode and anode electrode 50b, may stimulate at least one of the left and right side mental branch nerves 40 of the mouth and the intraoral device 52 may include more than one pair of anode and cathode electrodes 50a, 50b to stimulate both the left and right mental branch nerves 40 of the left and right sides of the mouth. It is also understood that more than two pairs of anode and cathode electrodes 50a, 50b may be carried by the intraoral device 52 to stimulate various areas along the mental branch nerves 40. In some embodiments, changing the phase of the modulating signal 70 to different mental branch nerves 40 may be used to enhance the stimulation of the mental branch nerves 40 by timing the delivery of the electrical stimulation to the multiple pairs of electrodes. In one embodiment, electrical simulation may be rotated across several electrodes placed across the target nerve, to maximize activation of that nerve without activating nearby nerves responsible for unwanted side effects.

In an alternative embodiment of the present invention, the electrodes 50 may be surface electrodes placed on an outer surface of the cheek, or subcutaneous electrodes inserted under the skin in the cheek region to electrically stimulate the buccal branch nerves of trigeminal nerves 42 located in an upper jawbone 45 region and the facial nerves below the cheek.

For example, the electrodes 50 are surface electrode pads placed externally on the patient's cheeks overlying the buccal branch nerves 26 to stimulate the buccal branch nerves 26 and/or facial nerves such as ophthalmic nerve (V1) 22 and mandibular nerve (V3) 26. The electrodes 50 may include a cathode electrode 50a and anode electrode 50b placed externally on the same cheek. Current flow between the anode and cathode electrodes 50a, 50b may provide electrical stimulation of the target nerve. A second set of anode and cathode electrodes 50a, 50b may be placed on the opposite cheek to stimulate the buccal branch nerves 26 and/or facial nerves of the opposite cheek. In one embodiment, non-invasive stimulation of the ophthalmic nerve (V1) 22 and mandibular nerve (V3) 26 has been found to cause increases in blood flow as seen in MRI 4D-flow imaging comparable to clinical methods of increasing blood flow using the drug dobutamine which increases heart rate contractility.

In another example, the electrodes 50 may be subcutaneous electrodes inserted beneath the epidermis within the patient's cheeks to stimulate the buccal branch nerves 26. The subcutaneous electrodes may be injectable electrodes 50 such as liquid metal electrodes injectable using a syringe 106 and withdrawable from the skin. The injectable electrodes 50 may include a cathode electrode 50a and anode electrode 50b placed under the skin. Current flow between the anode and cathode electrodes 50a, 50b may provide electrical stimulation of the target nerve. The injectable electrodes 50 may be injected into one or both cheeks to stimulate the buccal branch nerves 26.

In an alternative embodiment of the present invention, the electrodes 50 may be surface electrodes placed on an outer surface of the forehead above the eye, or subcutaneous electrodes inserted, e.g., by injection, under the skin in the forehead region, to electrically stimulate the supraorbital branches of the trigeminal nerve or trigeminal nerve fibers in a similar manner as described above with respect to electrodes 50 placed in the cheek region to electrically stimulate the buccal branch nerves of trigeminal nerves 42 located in an upper jawbone 45 region and the facial nerves below the cheek. Electrode placement can be improved by palpating the foramen to isolate and target the trigeminal nerve as it leaves the foramen above the orbit where it is most superficial and thus avoiding unwanted off-target activation.

In an alternative embodiment of the present invention, the electrodes 50 may be surface electrodes providing non-invasive stimulation of the auricular vagus at the cymba conchae/tragus. The electrodes 50 may be part of an earbud or headband supporting the electrodes 50 over the tragus.

In some embodiments, the stimulating electrodes 50 may be used to record electrophysiological signals in the brain, e.g., to detect changes in low frequency power brain waves that propagate outside the calvarium. The stimulating electrodes 50 may also be used to pick up other physiological signals of the patient such as heart rate and heart rate variability. Alternatively, separate electrodes from the stimulating electrodes 50 may be placed on the patient to record the electrophysiological signals in the brain and other physiological signals.

In some embodiments, the intraoral device 52 may include acceleration and gyroscopic sensors to detect a multitude of different patient outcomes, including sleep quality and patient activities of daily living (e.g., level of activity of the patient). The acceleration and gyroscopic sensors or other position sensors may also be used to determine patient positioning, i.e., patient posture, patient body position, for example, whether the patient is lying down or standing, and the like. The information may be used to determine sleep, activity, and position trends of the patient and/or the patient data of multiple patients may be aggregated to determine trends in the patient population.

The electrode 50 placement and intraoral device 52 may be as described in U.S. Pat. No. 11,395,914, titled “Penetration of Cerebral Spinal Fluid into the Brain Parenchyma using Temporally Pattered Neuromodulation,” filed Jul. 22, 2020, assigned to the present applicant and hereby incorporated by reference.

Other methods for modulating glymphatic clearance described herein may include any appropriate form of stimulation of the nerves. Examples of electrical stimulation modalities that may be used as described herein include, without limitation, peripheral nerve stimulation (e.g., vagus nerve stimulation and/or carotid sinus nerve stimulation), transcranial direct current stimulation (tDCS), deep brain stimulation (DBS), cortical stimulation, spinal cord stimulation (SCS), transcranial/transdermal magnetic stimulation (TMS), focused ultrasound, infrared stimulation, optogenetic activation, genetic modification to enhance sensitivity and specificity of the nerve to stimulation with a light source (optogenetics), and use of intravascular electrodes.

### Synergistic Electrical Stimulation Protocols

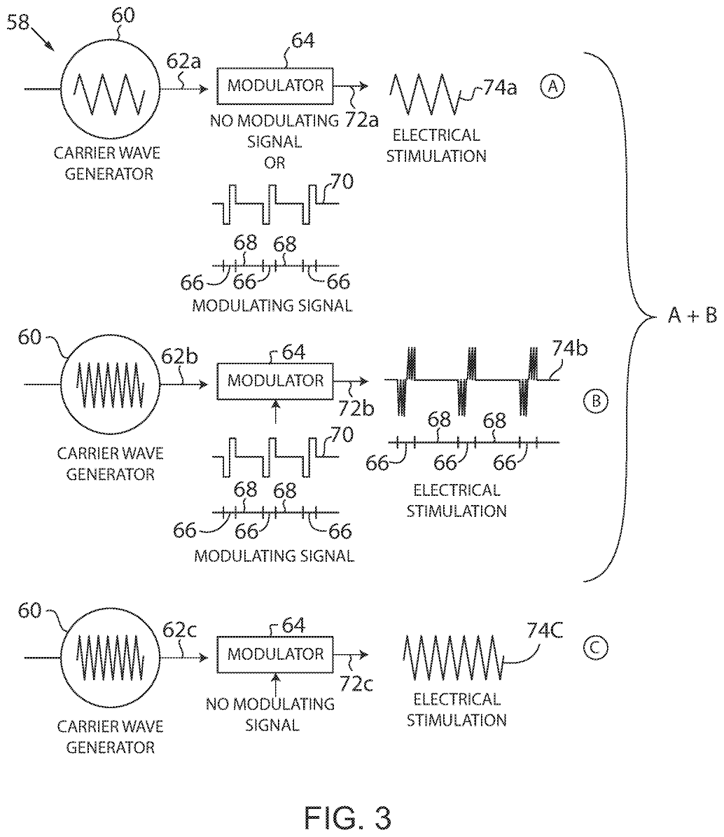

Referring now to FIGS. 2 and 3, a layered electrical stimulation protocol that (1) optimizes AQP4 mediated fluid exchange (i.e., fluid and molecular movement), (2) uses temporal patterning and stimulation parameters maximizing the cerebral vessel wall movement, (3) promotes phagocytic phenotype in glial cells to break down waste proteins, and (4) reduces neural activity to decrease fluid within the neuron, thereby increasing fluid in the extracellular space to reduce the resistance of the paranchymal pathway to fluid flow promoting clearance, are used to synergistically increase the CSF flow rate (i.e., flow speed) to the periarterial spaces to maximize waste clearance from the brain. The vessel wall movement may be defined as a change in the vessel diameter over time relative to a mean vessel diameter.

Electrical stimulation of the target nerves may be accomplished using an electrical stimulator 58 such as those commercially available from Tucker-Davis Technologies of Alachua, Florida or A-M Systems of Sequim, Washington. The electrical stimulator 58 may include a carrier wave generator 60 and a processor 102 being an electronic computer having a self-contained nonvolatile memory 103 holding an operating program 105 and necessary storage variables as will be described below. The nonvolatile memory 103 may comprise, for example, flash memory and/or read only memory, or other similar nonvolatile memory as context requires, which may store data values to be retained even in the absence of electrical power. The processor 102 may be a STM32 Nulceo board or PIC microcontroller as known in the art.

The processor 102 provides various inputs and output lines communicating, for example, with one or more stored programs 105 stored in non-transitory memory 103 and the carrier wave generator 60 to generate one or more carrier waves 62 at a carrier frequency and amplitude. The one or more carrier waves 62 are delivered to a modulator 64 modulating the one or more carrier waves 62 amplitude according to a modulating signal 70.

Referring specifically to FIG. 3, in one embodiment of the present invention, the processor 102 may communicate with one or more stored programs 105 stored in a non-transitory memory 103 and the carrier wave generator 60 to generate first, second, and third carrier waves 62a, 62b, 62c where the carrier waves 62a, 62b, 62c are delivered to the modulator 64, also communicating with the processor 102 and modulating amplitude of the second carrier waves 62b according to the modulating signal 70. In some embodiments, the first and third carrier waves 62a, 62c passes through the modulator 64 without modulation or alternatively may bypass the modulator 64 entirely if it is known that no modulation is needed to be applied to the first and third carrier wave 62a, 62c.

A first carrier wave 62a is delivered continuously at a low frequency between 0.1 Hz and 4.5 Hz and preferably centered around <1 Hz. The low frequency is desirably consistent with a frequency of electroencephalogram (EEG) brain activity during slow-wave sleep. Slow-wave sleep is phase 3 sleep and the deepest pages of non-rapid eye movement sleep. The slow-wave sleep is characterized by delta waves (between 0.5 Hz and 4.5 Hz). The first carrier wave 62a may have a current amplitude of less than 1000 microamps for invasive stimulation and less than milliamps for non-invasive stimulation and a voltage controlled to achieve this current per current control known in the art.

The first carrier wave 62a will pass through the modulator 64 without amplitude modification or without significant amplitude modification, or alternatively, will bypass the modulator 64 entirely to provide a first continuously electrical stimulation 72a output that has an amplitude and continuous duty cycle that is the same or similar to the first carrier wave 62a. Therefore, the first carrier wave 62a may be delivered continuously during and after a patient's sleep state to encourage opening of the AQP4 channels.

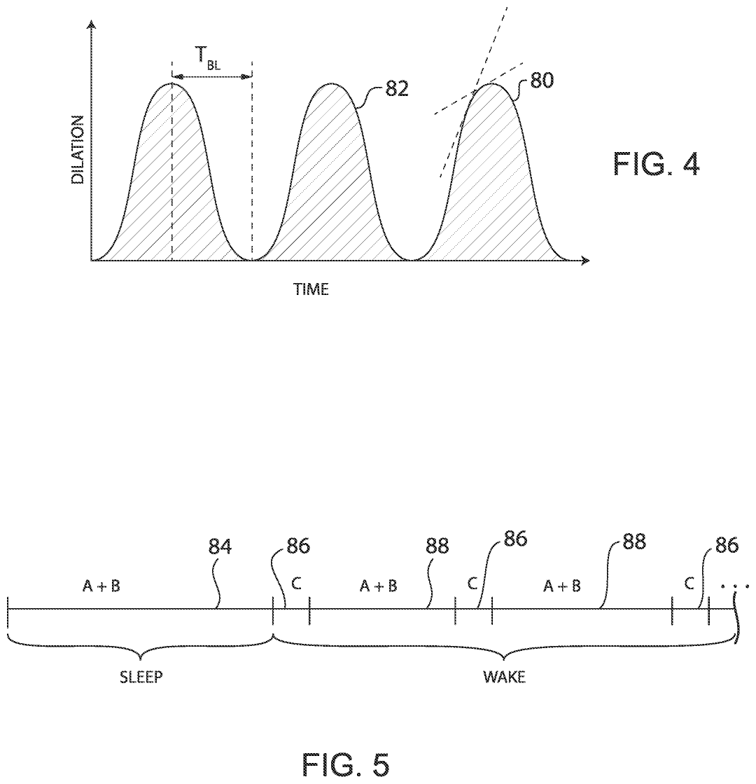

In some embodiments, the first carrier wave 62a is modulated and will pass through a modulator 64 to provide a temporal pattern of low frequency pulses 74a in the first electrical stimulation 72a output. In a first modulation period 66, e.g., “ON” pulse interval, there is minimal to no modification of the first carrier wave 62a and the first carrier wave 62a is allowed to pass without modifying or substantially modifying the amplitude of the signal (i.e., stimulation “ON” state) or has a comparatively higher amplitude, and in a second modulation period 68, e.g., “OFF” pulse interval, the modulator 64 will modify the first carrier wave 62a to reduce the electrical stimulation amplitude to substantially zero (i.e., stimulation “OFF” state) or has a comparatively lower amplitude. In this case, the modulating signal 70 may be a discontinuous waveform such as a biphasic pulse or square wave. As is understood in the art, signal modulation by the modulator 64 may provide an envelope of the peaks of the first carrier wave 62a, the latter being of much higher frequency than the modulating signal 70. Although the modulating signal is shown as a square wave in FIG. 3, the modulating signal 70 may also be a smooth curve as shown in FIG. 4. It is understood that during modulation, the modulator 64 may modify the first carrier wave 62a to an amplitude that differs between the first modulation period 66 and the second modulation period 68. For example, the amplitude of the first carrier wave 62a may be greater during the first modulation period 66 as compared to the second modulation period 68 and vice versa.

Referring briefly to FIG. 4, the stimulation parameters of the first carrier wave 62a may be empirically set to maximize the opening of AQP4 channels. While the inventors do not wish to be bound by a particular theory, it is believed that low delta frequency stimulation (e.g., to 4 Hz) delivered continuously during patient sleep or during patient wakeful state increases the fluid flux across AQP4 channels in the astrocytic endfeet surrounding descending arterioles in the brain. During the patient wakeful state, the low frequency stimulation may compensate for lowered fluid flux of AQP4 channels when slow-wave sleep frequencies are not naturally occurring.

As described in more detail below, this set point may be established, for example, by monitoring a set of patients during or immediately following delivery of the first electrical stimulation 72a to detect opening of AQP4 channels and CSF/ISF flow and adjusting the stimulation amplitude and frequency of the first carrier wave 62b to maximize the area 80 beneath the CSF/ISF flow curve 82.

Referring again to FIGS. 2 and 3, a second carrier wave 62b is delivered continuously at a higher frequency between 20 Hz and 75 Hz and between 20 Hz and 40 Hz and between 10 Hz to 15 Hz and preferably centered around 30 Hz. The second carrier wave 62b may have a current amplitude of less than 1000 microamps for invasive stimulation and less than 40 milliamps for non-invasive stimulation and a voltage controlled to achieve this current per current control known in the art.

The second carrier wave 62b will pass through the modulator 64 to provide a temporal pattern of high frequency pulses 74b in the second electrical stimulation 72b output. In a first modulation period 66 or “ON” pulse interval, there is no modification of the second carrier wave 62b and the second carrier wave 62b is allowed to pass without modifying the amplitude of the signal (i.e., stimulation “ON” state), and in a second modulation period 68 of “OFF” pulse interval, the modulator 64 will modify the second carrier wave 62b to reduce the electrical stimulation amplitude to substantially zero (i.e., stimulation “OFF” state). In this case, the modulating signal 70 may be a discontinuous waveform such as a biphasic pulse or square wave. As is understood in the art, signal modulation by the modulator 64 may provide an envelope of the peaks of the second carrier wave 62b, the latter being of much higher frequency than the modulating signal 70. Although the modulating signal 70 is shown as a square wave in FIG. 3, the modulating signal 70 may also be a smooth curve as shown in FIG. 4.

Referring briefly to FIG. 4, the stimulation parameters of the second carrier wave 62b and the modulating signal 70 may be empirically set to maximize arterial wall movement. While the inventors do not wish to be bound by a particular theory, it is believed that the accommodation or acclamation of the tissue to the stimulation effectively limits the clearance when continuous stimulation is provided as understood in the prior art. By interleaving higher frequency stimulation (of “ON” pulse interval 66) with periods of rest (of “OFF” pulse interval 68), vasoconstrictive/dilative recovery processes may be accommodated to maximize pulsatile motion and allow greater clearance in the long run. It has been found that continuous “ON” pulse intervals for long periods of time, e.g., 5 Hz or 10 Hz for greater or equal to 30 seconds, without periods of rest (“OFF” pulse interval) habituates blood flow changes (i.e., blood flow changes go back to baseline) and do not enable the desired outcomes in cerebral blood flow.

Ideally, the electrical stimulation parameters of the modulating signal 70 will provide a relaxation time (of “OFF” pulse interval 68) defined by the second carrier wave 62b being in an “OFF” state that is no less than the time to return to baseline (TBL) measured after brief periods of stimulation (of “ON” pulse interval 66) defined by the second carrier wave 62b being in an “ON” state. The ratio of the pulse duration of the “ON” stimulation to the total period of the ON/OFF waveform may be referred to as the “duty cycle” of the modulating signal 70.

As described in more detail below, this set point may be established, for example, by monitoring a set of patients during or immediately following delivery of a second electrical stimulation 72b to detect vessel wall movement and CSF/ISF flow and adjusting the amplitude and frequency of the second carrier wave 62b and temporal pattern of the modulating signal 70, i.e., stimulation time (stimulation “ON” state) and relaxation time (stimulation “OFF” state), to maximize the area 80 beneath the CSF/ISF flow curve 82. Thus, the dilation/constriction of arterial vessels at various modulating signal frequencies may be compared to maximize the area 80 under the curve 82 of FIG. 4, for example, slower, large amplitude changes in clearance (produced by prolonged carrier frequency stimulation) may be compared with faster, smaller amplitude changes in clearance (produced by shortened carrier frequency stimulation) to provide the greatest increases in CSF flow over time in the perivascular space. Similar comparisons may be done with respect to the spacing between stimulations provided by the relaxation period, i.e., the duty cycle.

While the stimulation parameters of the first electrical stimulation 72a and second electrical stimulation 72b may be empirically set separately, as described above, the first and second carrier waves 62a, 62b and modulating signal 70 may additionally or alternatively be empirically set when observed together so as to establish maximum synergistic effect of the first and second electrical stimulation 72a, 72b protocols to maximize the clearance of CSF. In this respect, the delivery of the stimulation parameters of the first carrier wave 62a and second carrier wave 62b as described above provides remarkable effects that are greater than the effects of each carrier wave 62a, 62b delivered separately and combined, which may be expected.

Referring again to FIGS. 2, 3 and 5, preferably during patient sleep or periods of slow-wave sleep, the first carrier wave 62a and second carrier wave 62b are overlaid or delivered simultaneously to provide a synergistic effect of greater consistent opening of AQP4 water channels found in the astrocytic endfeet, and movement of CSF flow in the parenchymal extracellular space through increased vessel wall movement, respectively, to create larger than expected CSF-ISF fluxes into and out of the brain or spinal cord as part of the CSF-ISF exchange.

The overlaid, simultaneously delivered first carrier wave 62a and second carrier wave 62b will be delivered during patient sleep but during patient wakeful state, will be periodically replaced by a third carrier wave 62c. The third carrier wave 62c is delivered continuously at high frequency gamma rhythms between 25 and 140 Hz and centered around 40 Hz. The third carrier wave 62c may have a current amplitude of less than 1000 microamps for invasive stimulation and less than 40 milliamps for non-invasive stimulation and a voltage controlled to achieve this current per current control known in the art.

The third carrier wave 62c will pass through the modulator 64 without modification or without substantial modification or alternatively will bypass the modulator 64. The third carrier wave 62c may be delivered continuously for periods of 15 minutes to 60 minutes between periods of administering the first carrier wave 62a and second carrier wave 62b simultaneously. The third carrier wave 62c may be delivered during, e.g., patient mid-sleep or during sleep or immediately following patient sleep during a wakeful state or continuously during wakeful state.

The delivery of the third electrical stimulation 72c enhances clearance of waste proteins between cycles of increased CSF-ISF fluxes into and out of the brain created by the first and second electrical stimulation 72a, 72b and thus enabling more clearance to occur.

Referring briefly to FIG. 4, the stimulation parameters of the third carrier wave 62c may be empirically set to promote a more phagocytic phenotype in glial cells to help break down waste biomolecules and misfolded proteins for subsequent clearance. While the inventors do not wish to be bound by a particular theory, it is believed that glial cells play a role in neurodegenerative conditions. Among glial cells, microglia and astrocytes play phagocytic roles by engulfing synapses, apoptotic cells, cell debris, and released toxic proteins. By administration electrical stimulation at gamma frequency (i.e., 20-50 Hz) for longer durations (e.g., 15 minutes to 60 minutes), the expression of pro-phagocytic genes and phenotypes in glial cells is increased, which promotes phagocytic states in glial cells. It is understood that flickering sound and light stimulation at gamma frequency (i.e., 20-50 Hz) may also enhance phagocytic states in glial cells and may be concurrently administered with electrical stimulation.

As described in more detail below, this set point may be established, for example, by monitoring a set of patients being during or immediately following delivery of a third electrical stimulation 72c to detect CSF/ISF flow, arterial dilation, and movement of metabolic waste within CSF including misfolded proteins, and adjusting the stimulation amplitude and frequency to maximize the area 80 beneath the CSF/ISF flow curve 82.

While the stimulation parameters of the third carrier wave 62c may be empirically set independently as described above, the third carrier wave 62c may additionally or alternatively be empirically set when administered with the first carrier wave 62a and second carrier wave 62b, and modulating signal 70 so as to establish maximum synergistic effect of the three electrical stimulation protocols to maximize the clearance of CSF. In this respect, the delivery of the stimulation parameters of the first carrier wave 62a and second carrier wave 62b together with the third carrier wave 62c as described provides remarkable effects that are greater than the effects of each carrier wave 62a, 62b, 62c delivered separately and added together which may be expected.

It is understood that in some embodiments, the third carrier wave 62c may be administered prior to the administration of the first carrier wave 62a, second carrier wave 62b, and modulating signal 70, and as an initial step in the stimulation protocol in order to induce phagocytosis prior to increasing vessel wall movement and CSF clearance with the first carrier wave 62a, second carrier wave 62b.

Referring again to FIGS. 1 and 2, the electrical stimulator 58 may communicate with the electrodes 50, for example, mounted on an intraoral device 52 to deliver the electrical pulses 74a, 74b, 74c of the electrical stimulation 72a, 72b, 72c, respectively, to the electrodes 50.

In one embodiment, the electrical stimulator 58 may be external to the intraoral device 52 and communicate wirelessly with the electrodes 50 on the intraoral device 52. In an alternative embodiment, the electrical stimulator 58 may be external to the intraoral device 52 or incorporated or molded onto the intraoral device 52 to communicate with the electrodes 50 on the intraoral device 52 via a wired connection.

In an alternative embodiment, the electrical stimulator 58 may also communicate with electrodes 50 which are surface electrodes or subcutaneous electrodes or other electrical stimulation modalities as previously described.

It is contemplated that there may be multiple electrodes 50 positioned at different locations on the patient to stimulate different target nerves. Each of the multiple electrodes 50 may deliver different electrical pulses 74a, 74b, 74c and at different times in order to optimize maximum synergistic effect of the three electrical stimulation protocols to maximize the clearance of CSF. This may be desired based on different target nerves responding differently to the electrical stimulation 72a, 72b, 72c and the respective frequencies. For example, the first carrier wave 62a and second carrier wave 62b may be delivered to a first target nerve that responds well to increasing arterial wall movement and the third carrier wave 62c may be delivered to a second target nerve that responds well to inducing phagocytosis. Although the inventors do not wish to be bound by a particular theory, it is believed that certain target nerves are better at modulating arterial wall movement while others are better at modulating astrogliotic activity.

Real-time sensing of optimized electrode target engagement may be used to determine an optimal target location for brain activation and to monitor and provide maximum effect on CSF/ISF flow as further discussed below.

### Example 1: Synergistic Stimulation Protocol

Referring to FIG. 5, the first and second electrical stimulation 72a, 72b may be delivered simultaneously and continuously for a first period 84 that is consistent with a patient's sleep duration or for at least one non-rapid eye movement (NREM) sleep cycle. The first period 84 may be at least 30 minutes or at least 1 hour and at least 2 hours and at least 3 hours and at least 4 hours and at least 5 hours and at least 6 hours and at least 7 hours and at least 8 hours and between 1 to 8 hours in duration consistent with at least one NREM sleep cycle and during periods of deep wave sleep.

The third electrical stimulation 72c may replace the first and second electrical stimulation 72a, 72b delivered simultaneously, to be delivered periodically (regular occurring intervals) and continuously for a second period 86 following patient sleep. It is understood that due to the high frequency of the third carrier wave 62c, delivery of the third electrical stimulation 72c may disrupt a patient's sleep, therefore, it is preferably delivered during a wakeful state of the patient. In some embodiments, a period of the third electrical stimulation 72c may be delivered mid-sleep or sometime during the sleep period to help promote clearance during sleep state.

The third electrical stimulation 72c is delivered for a second period 86 which may be at least 15 minutes and at least 30 minutes and at least 45 minutes and at least 1 hour duration and less than 30 minutes and less than 45 minutes and less than 1 hour and may be a period of 15 minutes to 60 minutes. Following the delivery of the third electrical stimulation 72c, delivery of the first and second electrical stimulation 72a, 72b may resume for a third period 88 during a patient's wakeful state until being interrupted by the subsequent repeated second period 86 of the third electrical stimulation 72c. The second period 86 and third periods 88 occur during the wakeful state of the patient and may repeat for a duration of at least 1 hour and at least 2 hours and at least 3 hours and at least 4 hours and at least 5 hours and at least 6 hours and at least 7 hours and at least 8 hours and at least 9 hours and at least 10 hours and at least 11 hours and at least 12 hours and between 1 to 16 hours and consistent with a desired length of treatment during the patient's wakeful state.

The delivery of the third electrical stimulation 72c will be delivered periodically (at regular occurring intervals), for example, every 30 minutes, every 60 minutes, every 90 minutes, every 120 minutes, or every 30 to 120 minutes in changing time intervals that may increase over time. The second period of time 86 may be repeated at least twice or at least three times or at least four times or at least five times or continuously during the patient's wakeful state and during the concurrent but separate administration of the first and second electrical stimulation 72a, 72b during the patient's wakeful state.

In some embodiments, the duration of the third electrical stimulation 72c of the second period of time 86 may be reduced over time for subsequent deliveries of the third electrical stimulation 72c, for example, when less waste proteins need to be cleared after subsequent deliveries of the third electrical stimulation 72c. Also, the amount of time between the third electrical stimulation 72c may be increased over time for subsequent deliveries of the third electrical stimulation 72, for similar reasons.

### Optimizing Target Nerve and Brain Engagement

The present invention provides real-time sensing, monitoring, and indication of target nerve engagement and corresponding brain activity using non-invasive and minimally invasive sensing systems 108. Further, high impedance or poor electrode signal issues can be detected and troubleshooted prior to initiating the electrical stimulation protocol treatment also using non-invasive and minimally invasive sensing systems 108.

Optimal target engagement depends on several electrode parameters, such as electrode design, electrode placement, and stimulation waveform parameters delivered to the electrode to help improve on-target engagement and minimize off-target activation. It is desirable to provide direct measures of target nerve engagement or representative measure in real-time to provide set point guidelines for electrode parameters in terms of the positioning of the stimulating electrodes and electrical stimulation modulation waveforms, as were described above with respect to electrical stimulation protocols for the first carrier wave 62a, second carrier wave 62b, and modulating signal 70, delivered in alternating fashion with the third carrier wave 62c, for individual patients or classes of patients.

Similarly, the optimal deep brain activation depends on electrode parameters as well as various stimulation parameters, such as polarity, stimulation duration, intensity level, and the patient's brain state. It is desirable to observe target brain engagement in real-time to provide set point guidelines for electrical stimulation protocols in terms of the location of the stimulating electrodes and modulation duration (i.e., temporal pattern and duty cycle) and intensity level (i.e., frequency and amplitude) of the electrical stimulation for individual patients or classes of patients. Specifically, the electrical stimulation protocols will be optimized to determine the optimal amplitude and frequency of the carrier waves 62a 62b, 62c, and amplitude, frequency, and duty cycle of the modulating signal 70, as described above with respect to the simultaneously delivered electrical stimulation 72a, 72b and alternating electrical stimulation 72c to the electrodes 50.

Target nerve engagement and deep brain engagement may be monitored using known recording methods and imaging modalities such as measuring changes in compound action potentials induced by electrical stimulation, changes in electroencephalogram (EEG) recordings induced by electrical stimulation, and changes in single event related potentials induced by electrical stimulation. These recording and neuroimaging methods may be useful to locate and quantify the effects of electrical stimulation on deep brain activity for target peripheral nerves. Neuroimaging method that may be used with the present invention include known uses of electric, magnetic, and other techniques to detect the properties of function, structure, or changes in the brain in terms of temporal (i.e., functional imaging) and spatial localization (i.e., structural and functional imaging).

It is understood that other physiological factors may also be monitored to determine the effectiveness of target nerve engagement or optimal deep brain activation indirectly, such as observed changes to heart rate, respiratory rate, fractional anisotropy, or presence of certain biomarkers in the patient's blood or saliva as further described below. For example, magnetic resonance imaging (MRI) and positron emission tomography (PET) can provide information on structural, hemodynamic and chemical changes associated with electrical stimulation. Physiological factors can be detected using, for example, neuroimaging techniques (e.g., panoramic x-ray, computerized topography (CT) scan, diffuse optical imaging (DOI), event-related optical signal (EROS), magnetic resonance imaging (MM), functional magnetic resonance imaging (fMRI), magnetoencephalography (MEG), positron emission tomography (PET), single-photon emission computed tomography (SPECT), and cranial ultrasound), cognitive function testing (e.g., learning tests and memory tests), motor function testing, sensory function testing, biopsy, CSF testing, blood testing and/or genetic testing, acceleration and/or gyroscopic sensing, and patient position sensing.

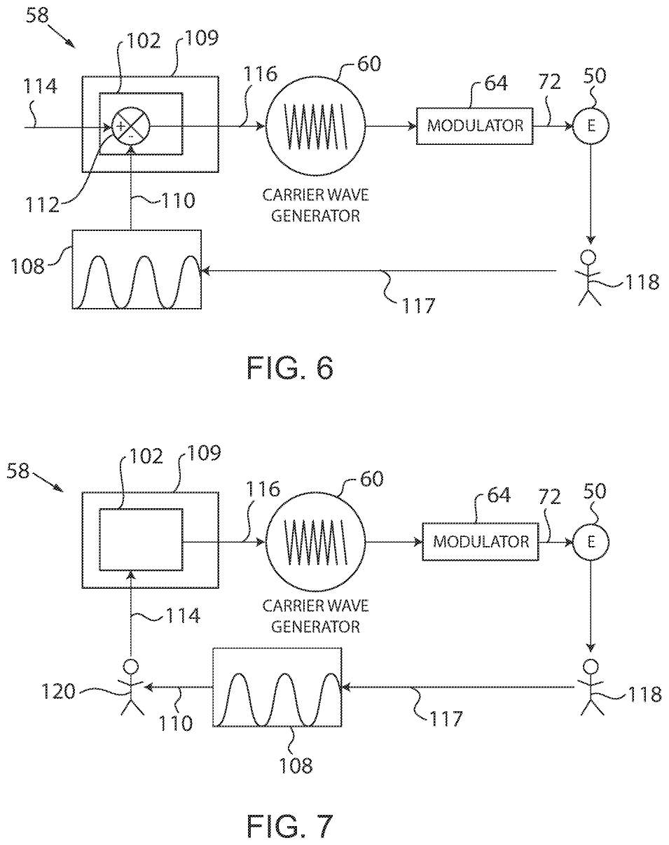

The following are exemplary embodiments of non-invasive and minimally invasive recording and neuroimaging methods that may be used to detect target nerve engagement and deep brain activation to optimize CSF clearance in a closed loop or open loop operated sensing systems 108 as illustrated in FIGS. 6 and 7.

### Example 2: Measuring Evoked Compound Action Potential

An electrically evoked compound action potential (eCAP) is a measure of the electrical response from a peripheral nerve to electrical stimulation and represents the synchronous firing of a proportion of electrically stimulated nerve fibers. The use of eCAPs can provide direct insight into the electrophysiology of the electrical stimulation and can assist with determining the effectiveness of the electrical stimulation.

The eCAP techniques of recording neural activity generated by electrical stimulation rely on a minimally invasive stimulus applied directly to the peripheral nerve, or indirectly non-invasively applied to the surrounding tissue of the target nerve, to evoke a compound action potential that can be recorded by the operator. The stimulus simultaneously and synchronously recruits many different fibers, thus, the response is referred to as a compound action potential because it is the sum of the many potentials recruited. The summation and synchronous generation of action potentials increases the amplitude of the response and thus makes neural activity detection more detectable.

The electrical activity response of the peripheral nerve neurons can be recorded using minimally invasive or non-invasive electromyography (EMG) to assess the integrity of the nerve being stimulated and the impact of electrical stimulation on the peripheral nerve. The conduction velocity and propagation times of the action potential can assist the operator in determining the diameter of the fiber and the nerve fiber type providing useful knowledge that can assist with optimizing target location and electrical stimulation protocols of the peripheral nerve. For example, A-beta fibers are 6-12 μm in diameter and have a conduction velocity of 33-75 m/s, A-delta fibers are 1-5 μm in diameter and have a conduction velocity of 3-30 m/s, and C-fibers are μm in diameter and have a conduction velocity of 0.5-2 m/s. Therefore, activation of different fiber types can be determined.

In one embodiment of the present invention, the vagal nerve is responsible for carrying information to and from the brain. Recording eCAPs can be measured along the vagal nerve pathway. For example, A-beta, A-delta, and C fibers may be activated during vagal nerve stimulation. The latency of the response can help determine which nerve fiber type is being stimulated along the vagal nerve pathway. The real-time feedback control from the measured compound action potentials can be used to determine how to activate a desired proportion of A-beta, A-delta, or C-fibers most efficiently over time.

### Example 3: Microneurography

Microneurography is a method that provides visualization of nerve impulses that are conducted in peripheral nerves. Microelectrodes (i.e., needle electrodes) may be inserted percutaneously into the nerve, or surface electrodes may be placed non-invasively on the skin close to the target nerve. An electrical stimulation is applied to the peripheral nerve to elicit a single compound action potential representing the target nerve output. The electrical stimulation may be elicited closer to the nerve trunk to ensure activation of the superficial sensory receptors and connected nerve ending.

Ultrasound may be used to guide the placement of the electrodes by palpating the tissue to distinguish the nerve from the surrounding tissue and by imaging the target nerve to help guide the position of the microelectrodes or surface electrodes to receive an adequate electrical signal, e.g., with good signal-to-noise ratio. Ultrasound may offer higher resolution imaging and is more cost effective than other imaging modalities. The ultrasound transducer may be placed non-invasively or beneath the tissue within a surgical pocket.

By recording the impulse activity of individual nerve fibers in real-time, optionally with the assistance of ultrasound, correlations can be made between electrical stimulation and neural activity effects to determine optimal electrode location with respect to specific nerve fibers.

### Example 4: Electroencephalography and Magnetoencephalography

Electroencephalography (EEG) and magnetoencephalography (MEG) are non-invasive neuroimaging methods for measuring brain activity in response to an electrical stimulation. Recording surface electrodes placed on the brain can detect small electrical or magnetic activity changes that result from neural activity within the brain due to electrical stimulation of the peripheral nerve. EEG measurements are based on electrical potential differences between different recording electrodes on the scalp. MEG measurements are based on magnetic fields generated by electric currents in the brain.

During electrical stimulation of a peripheral nerve, the electrical stimulation will generate an evoked EEG potential in the brain which can be recorded by the EEG or MEG recording electrodes or sensors (i.e., magnetometers and gradiometers). Exemplary features extracted from EEG and MEG include the location of activation of the event-related potential, coherence, connectivity, and power spectra in different frequency bands (e.g., alpha, beta, theta, gamma, etc.).

The power spectrum is widely used as the monitoring index to examine the EEG modulation effects. A commonly applied algorithm is a short-time Fourier transform and wavelet transform. The power spectrum can describe an EEG signal's power distribution by frequency and time.

In one embodiment of the present invention, electrical stimulation can induce oscillation within the brain at EEG power dynamics within desired alpha and gamma frequency bands (e.g., 2 Hz and 40 Hz targets). By detecting the desired power spectra and frequency bands in the brain, optimal “treatment windows” can be detected during which electrical stimulation may have optimal effect.

### Example 5: Electrical Stimulation Levels and Impedance Checks

Non-invasive measurements of autonomic nerve activity may be used to troubleshoot electrode impedance or poor electrode connections. In this respect, physiological measurements may be used to determine good connectivity of the electrodes and proper functioning of the system. The electrical stimulation levels may also be checked by measuring the level of brain activity elicited by the peripheral nerve innervation.

Non-invasive measurements of autonomic nerve activity may be performed including blood oxidation, blood pressure, galvanic skin response, pulse rate, respiratory rate, heart rate, and the like. Electrodes may be used to record electrophysiological signals to detect changes in low frequency power brain waves that propagate outside the calvarium, for example, electrocardiogram and photoplethysmography. Electrodes may also be used to pick up heart rate and heart rate variability. The electrodes may be used to make measurements during periods of stimulation and/or during periods of no stimulation.

It is understood that other physiological factors may also be monitored to determine the effectiveness of stimulation parameters, such as changes to heart rate, respiratory rate, fractional anisotropy, or presence of certain biomarkers in the patient's blood or saliva as further described below. These physiological factors may be measured using, for example, neuroimaging techniques (e.g., panoramic x-ray, computerized topography (CT) scan, diffuse optical imaging (DOI), event-related optical signal (EROS), magnetic resonance imaging (MM), functional magnetic resonance imaging (fMRI), magnetoencephalography (MEG), positron emission tomography (PET), single-photon emission computed tomography (SPECT), and cranial ultrasound), cognitive function testing (e.g., learning tests and memory tests), motor function testing, sensory function testing, biopsy, CSF testing, blood testing and/or genetic testing. Acceleration and gyroscopic sensing and other position sensing may be used to measure patient position, and the like.

### Optimizing Stimulation Parameters to Increase CSF Flow and Glymphatic Clearance

The present invention provides real-time sensing of CSF clearance and glymphatic clearance using non-invasive and minimally invasive sensing systems 108 detecting blood flow (perfusion), brain organization, and brain activity during or after stimulation.

As noted above, the processor 102 of the carrier wave generator 60 executing one or more stored programs 105 stored in non-transitory memory 103 may communicate with the sensing system 108 to automatically determine a frequency in which AQP4 channels are opened, vessel wall movement is maximized, and the promotion of phagocytic phenotype in glial cells by providing variations in the above described parameters and monitoring clearance appropriately. Similarly, the processor 102 may automatically determine a minimum time duration of the electrical pulses required to provide maximum effect.

The CSF/ISF flow or measurements representative of CSF/ISF flow may be monitored using known sensing systems 108, for example, imaging modalities such as computerized topography (CT), magnetic resonance imaging (MM), functional magnetic resonance imaging (fMRI), positron emission tomography (PET), transcranial ultrasound, single-photon emission computed tomography (SPECT) during electrical stimulation, as further described below.

It is understood that other physiological factors may also be monitored to determine the effectiveness of stimulation parameters, such as changes to heart rate, respiratory rate, fractional anisotropy, or presence of certain biomarkers in the patient's blood or saliva as further described below. These physiological factors may be measured using, for example, neuroimaging techniques (e.g., panoramic x-ray, computerized topography (CT) scan, diffuse optical imaging (DOI), event-related optical signal (EROS), magnetic resonance imaging (MM), functional magnetic resonance imaging (fMRI), magnetoencephalography (MEG), positron emission tomography (PET), single-photon emission computed tomography (SPECT), and cranial ultrasound), cognitive function testing (e.g., learning tests and memory tests), motor function testing, sensory function testing, biopsy, CSF testing, blood testing and/or genetic testing.

Referring again to FIG. 4, the set points for the first, second and third carrier waves 62a, 62b, 62c and modulating signal 70 may be determined by observing the effects of each stimulation individually, or in combined effect with each other, e.g., the first and second carrier waves 62a, 62b applied together, or the first, second, and third carrier waves 62a, 62b, 62c applied together, to provide a desired synergistic effect with respect to opening AQP4 channels, increasing vessel wall movement, promoting phagocytic phenotype in glial cells, and increasing CSF/ISF flow and clearance.

The preferred timing of delivery of the first, second and third carrier waves 62a, 62b, 62c and corresponding electrical stimulation 72a, 72b, 72c may also be determined by sensing a preferred “therapeutic window” for electrical stimulation. For example, by monitoring a set of patients being scanned using imaging modalities described herein, periods of greater opening of AQP4 channels occurring during, for example, high frequency gamma rhythms of the brain sensed using, e.g., EEG or brain imaging modalities, may be detected and thus electrical stimulation preferably delivered during these “therapeutic window” periods of time to maximize clearance.

The following are exemplary embodiments of non-invasive and minimally invasive methods for optimizing electrical stimulation parameters and timing of delivery by observing physiological changes in the brain using closed loop and open loop operated sensing systems 108.

### Example 6: Brain Perfusion and Diffusion Imaging

A measurement of perfusion or blood flow to the brain can be used as an indicator of CSF flow changes due to electrical stimulation of the peripheral nerve. Imaging machines that can be used to detect blood flow and may use dynamic imaging of movement of a contrast agent to measure perfusion include computerized topography (CT) scanner, magnetic resonance imaging (MM) scanner (e.g., conducting dynamic susceptibility contrast (DSC) MM, blood-oxygen-level-dependent fMRI, arterial spin labeling fMRI, and the like), positron emission tomography (PET) scanner, transcranial ultrasound, and single-photon emission computed tomography (SPECT) scanner.

The following are examples of imaging methods for detecting perfusion or blood flow in the brain.

6a. Blood-Oxygen-Level-Dependent Functional Magnetic Resonance Imaging

Magnetic resonance imaging (MM) and functional magnetic resonance imaging (fMRI) use echo waves to discriminate among grey matter, white matter, and cerebrospinal fluid to show flow of blood in the brain. fMRI scans are a series of scans that measure the structure and functional activity of the brain through multiple images.

Blood flow is very sensitive to changes in neural activity, therefore, blood flow changes can be used as a sensitive indicator of underlying neural activity changes. The blood-oxygen-level-dependent (BOLD) imaging is a non-invasive fMRI imaging technique for measuring and mapping localized networks of activity throughout the brain via measurement of blood flow that can be detected based on differential magnetic susceptibility.

The BOLD response in target areas of the brain can be measured in response to electrical stimulation of peripheral nerves and can be compared with evoked electrical recordings from stimulation of other nerves. Various electrical stimulation parameters may also be investigated such as stimulation frequency, stimulation current, and precise electrode location. BOLD imaging can help to optimize target location to an individual nerve fiber.

In one embodiment of the present invention, one can use BOLD imaging to confirm the optimal target location and stimulation protocol in real-time by observing perfusion in the prefrontal cortex and hippocampus associated with neurocognitive deficits, e.g., in Alzheimer's disease, during electrical stimulation of a peripheral nerve.

6b. Arterial Spin Labeling Functional Magnetic Resonance Imaging

Arterial spin labeling (ASL) imaging is a non-invasive fMRI technique for measuring cerebral blood flow and which uses magnetically “labeled” arterial blood water protons as an endogenous tracer which is observed by fMRI as it travels to the brain. The ASL images are compared to images from other nerves. Various electrical stimulation parameters may also be investigated such as stimulation frequency, stimulation current, and precise electrode location.

In one embodiment of the present invention, one can use ASL imaging to confirm the optimal target location and stimulation protocol in real-time by observing perfusion in the prefrontal cortex and hippocampus associated with neurocognitive deficits, e.g., in Alzheimer's disease, during electrical stimulation of a peripheral nerve.

6c. Positron Emission Tomography

Positron emission tomography (PET) scans measure levels of sugar glucose in the brain in order to measure where neural firing is taking place. As part of the PET scan, a tracer substance attached to radioactive isotopes is injected into the blood. The tracer is most often injected into a vein within the patient's hand or arm. The tracer will then collect into areas of the patient's body that have higher levels of metabolic or biochemical activity. When parts of the brain become active, blood (which contains the tracer) is sent to deliver oxygen. This creates visible spots, which are then picked up by detectors and used to create multiple images or a video image of the brain. Thus, the PET scan can monitor cerebral blood flow in the brain via sugar glucose level.

The PET images are typically combined with CT or MM and are called PET-CT or PET-MRI scans. Increased cerebral blood flow in key areas of the brain indicate desirable induced changes in the brain.

In one embodiment of the present invention, one can use PET scans to confirm the optimal target location and stimulation protocol in real-time by observing perfusion in the prefrontal cortex and hippocampus associated with neurocognitive deficits, e.g., in Alzheimer's disease, during electrical stimulation of a peripheral nerve.

6d. Diffusion Tensor Imaging

The diffusion of water molecules can generate contrast in MM images. Diffusion tensor imaging (DTI) is an MM technique that uses anisotropic diffusion of water molecules and other molecules to estimate the axonal (white matter) organization of the brain. It is based on diffusion differences in molecules as they travel along the body's tissues depending on type, integrity, architecture, and presence of barriers. Thus, DTI can provide information about the magnitude of molecule diffusion, the degree of anisotropy, and the orientation of directional diffusion. Thus, DTI can monitor cerebral blood flow in the brain via measurement of molecule diffusion.

In one embodiment of the present invention, one can use DTI to confirm the optimal target location in real-time and stimulation protocol by observing diffusion in the prefrontal cortex and hippocampus associated with neurocognitive deficits, e.g., in Alzheimer's disease, during electrical stimulation of a peripheral nerve.

### Example 7: Measuring Pupillary Dilation and Other Parasympathetic Responses

Electrical stimulation of peripheral nerves elicits responses mediated by parasympathetic reflex mechanisms. Pupillometers can be used to detect pupil dilations following electrical stimulation of peripheral nerves to evaluate evoked cortical neuromodulation. Electrical stimulation of peripheral nerves has been shown to dilate the pupil with the degree of pupil dilation dependent on both frequency and intensity of the stimulation. Optimal stimulation parameters may occur when current leakage and off-target effects are minimized and the extent of pupil dilation tracks electrical stimulation evoked basal-forebrain cholinergic axon activity in neocortex. Thus, pupil dilation is a sensitive measurement of the real-time, titratable effects of electrical stimulation on brain state.

Further, electrical stimulation of peripheral nerves elicits other responses mediated by parasympathetic reflex mechanisms. These responses include vasodilation in the orofacial area (e.g., lower lip, gingiva, palate, and tongue) and salivary or lacrimal secretions from the submandibular, parotid, and lacrimal glands. Sensors detecting salivary biomarkers may indicate a change to CSF flow selected from at least one of the following: amyloid beta peptide, tau protein, lactoferrin, alpha-synuclein, DJ-1 protein, chromogranin A, huntingtin protein, DNA methylation disruptions, and micro-RNA. Additionally, oral vasodilation may be determined directly from sensors placed in the oral cavity, i.e., pulse oximetry sensors located in an intraoral device.

In one embodiment of the present invention, one can use vasodilation and salivary or lacrimal secretions such as pupillary dilation to confirm the optimal target location and stimulation protocol in real-time by observing parasympathetic reflexes during electrical stimulation of a peripheral nerve.

### Real-Time Titration of Electrical Stimulation

Referring to FIGS. 6 and 7, a controller 109 of the electrical stimulator 58, for example, containing the processor 102 executing the programming 105 stored in memory 103 according to desired CSF clearance may communicate with the sensing systems 108 of the present invention to control the electrical stimulator 58 to deliver a desired signal to the stimulating electrodes 50. In some embodiments, the stimulating electrodes 50 may also be used as recording electrodes of the sensing system 108 to sense the biological changes delivered to the sensing systems. The processor 102 may also communicate with a display screen to provide information on a display screen and exposed on the outside of the electrical stimulator 58, and various input buttons allowing control of electrical signal parameters, for example, frequency, amplitude, duty cycle, and the like to be inputted by the operator according to techniques well known in the art.

Referring specifically to FIG. 6, in one embodiment of the present invention, a “closed loop” system feedback control uses the sensing system 108 and medical data 110 received from the sensing system 108 to control the electrical stimulation parameters of the electrical stimulator 58 to modulate CSF clearance. The sensors of the sensing system 108 and the medical data 110 from the sensing system 108 can be used within a closed loop feedback control to automatically adjust the electrical stimulation parameters of the electrical stimulator 58 to maximize or minimize CSF clearance. In this respect, the closed loop feedback control may be used for optimizing electrical stimulation protocols.

The medical data 110 may be imaging or sensor data such as event related potentials induced by electrical stimulation, EEG or MEG signals, blood oxidation, blood pressure, galvanic skin response, pulse rate, respiratory rate, heart rate, and data from computerized topography (CT), magnetic resonance imaging (MM), functional magnetic resonance imaging (fMRI), positron emission tomography (PET), transcranial ultrasound, single-photon emission computed tomography (SPECT), and the like, and as described above.

In one embodiment of the present invention, the sensing system 108 may provide medical data 110 received from biological signals 117 of a patient 118, for example, communicating through electrodes 50, at a summing junction 112 implemented through software in the memory 103 executed by the processor 102. The summing junction 112 also receives a command signal 114, for example, provided through the display screen or input button by input from the operator and describing a desired CSF flow rate, CSF clearance level, or other value indicative of CSF flow rate or CSF clearance level. For example, the command signal 114 may indicate a desire to maximize CSF flow rate or clearance and therefore the area 80 under the curve 82 may be maximized by the processor 102 as described above with respect to FIG. 4. In contrast, the command signal 114 may indicate a desire to minimize CSF flow rate or CSF clearance and therefore intervention such as electrical stimulation from the electrical stimulator 58 may be halted.

An output from the summing junction 112 provides an error signal 116 to the controller 109 of the electrical stimulator 58 driving the carrier wave generator 60 and modulator 64 to adjust the frequency and amplitude of the carrier wave 62 and/or the frequency, amplitude, and duty cycle of the modulating signal 70 to adjust the electrical stimulation 74 according to the command signal 114 and thereby providing a desired CSF flow rate, CSF clearance level, or other value indicative of CSF flow rate or clearance level. This error signal 116 provides feedback that eliminates the need for precise monitoring of the operation of the electrical stimulator 58 by a human operator. It is also understood that some minimal human operator intervention may provide safety checks within the closed loop system, for example, requiring the operator to approve or authorize adjustments to the electrical stimulation 74 before changes are made.

In one embodiment of the present invention, the processor 102 executing the programming 105 stored in memory 103 holding programming 105 may detect the area 80 beneath a CSF/ISF flow curve 82 as seen in FIG. 4, and thus may be programmed to adjust stimulation protocols to maximize the area 80 beneath the CSF/ISF flow curve 82 when maximum CSF flow is desired.

In an alternative embodiment of the present invention, the processor 102 executing the programming 105 stored in memory 103 holding programming 105 may detect CSF flow trends by detecting CSF flow as a function of time as seen in FIG. 4. For example, higher positive slopes will indicate CSF clearance is increasing and therefore maximum clearance levels have not yet been reached and lower positive slopes will indicate CSF clearance is slowing down and therefore is close to reaching maximum CSF clearance. A slope that is close to zero will indicate that CSF clearance is at or close to maximum clearance levels. Therefore, programming 105 may indicate to the electrical stimulator 58 to continue delivering electrical stimulation until maximum levels of clearance have been reached.

Referring specifically to FIG. 7, in an alternative embodiment of the present invention, an “open loop” or “human-in-the-loop” feedback control may include a human intervention to control electrical stimulation parameters by giving the medical data 110 to a human operator 120 and allowing the operator 120 to manually adjust the electrical stimulation parameters to modulate CSF clearance. The “human-in-the-loop” feedback control may be used to allow the operator to receive medical data 110 and change the location of activation or electrical stimulation protocol for optimal target nerve activation for maximizing CSF clearance. In this respect, human-in-the-loop feedback control may be used for optimizing target nerve engagement.

In one embodiment of the present invention, the sensing system 108 may provide medical data 110, received from biological signals 117 of a patient 118, for example, communicating through electrodes 50, and provided to a human operator 120, for example, provided through a display screen of the sensing system 108 or electrical stimulator 58 and viewable by the human operator 120. The processor 102 may provide the human operator 120 raw data or computer analyzed data assisting with interpretation of the medical data 110. The human operator 120 will receive the medical data 110 and manually adjust the command signal 114 indicating the frequency and amplitude of the carrier wave 62 and/or the frequency, amplitude, and duty cycle of the modulating signal 70 adjusted by the human operator 120 according to the desired CSF flow rate, CSF clearance level, or other value indicative of CSF flow rate or clearance level and thereby providing an optimal electrical stimulation protocol. This human operator 120 allows for more human oversight to operation and adjustment of the electrical stimulator 58 than closed loop systems.

The above described methods may be used to treat patients with, for example, depression, anxiety and epilepsy by increasing the influx of CSF into the brain parenchyma. It has been found that an increase in CSF into the brain parenchyma further dilutes endogenous concentrations of neurochemical transmitters/bioactive molecules and reduces ephaptic (non-synaptic) coupling implicated in abnormal circuit behaviors associated with multiple disorders of the nervous system, for example, anxiety disorders, epilepsy, Alzheimer's disease, and Parkinson's disease.

It is understood that the present invention is not limited to the treatment of traumatic brain injury/chronic traumatic encephalopathy, epilepsy, Alzheimer's disease, and Parkinson's disease and the like and may also be used to treat other conditions and disorders such as hydrocephalus caused by a buildup of CSF in the brain parenchyma by increasing the clearance of CSF through the brain. Also, clearance of orally administered drugs that cross the blood brain barrier, or drugs/biomolecules that are infused via an injection/catheter, can be modulated by changing the CSF flow rate.

Electrical stimulation of the nerves as described above has been found to induce neuroplasticity or cortical plasticity and introduce and modify brain wave oscillation frequency useful for treating neuro-psychiatric disorders. For example, brain wave oscillations may be increased to natural brain wave frequencies, e.g., 8 to 13 Hz, which may be lower in older adults experiencing memory difficulties, and activation of circuitry through the trigeminal sensory nuclei to create broad neurochemical changes in the brain mediated by cross connectivity to the nucleus of the solitary tract (NTS) to enhance plasticity in many conditions such as stroke and tinnitus. The NTS has inputs to locus coeruleus, raphae nucleus, and nucleus basalis which are responsible for most norepinephrine, serotonin, dopaminergic, and cholinergic projections to the rest of the brain.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “an electronic computer” and “a processor” or “the microprocessor” and “the processor,” can be understood to include one or more of these devices that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

References to “a processor” should be understood to include electronic computers, microprocessors, microcontrollers, FPGA devices, ASIC devices and similar programmable or program defined electronic circuits and collections of such devices that can communicate in a stand-alone and/or a distributed environment(s), and can thus be configured to communicate via wired or wireless communications with other processors. Furthermore, references to memory, unless otherwise specified, can include one or more processor-readable and accessible memory elements and/or components that can be internal to the processor or external to the processor and accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. An electrical stimulation device for modulating function of a glymphatic system or meningeal lymphatic system of a human patient comprising:
at least one electrode configured to stimulate a cranial nerve of the human patient;
an electrical generator configured to generate at least one carrier wave having a first carrier amplitude and a first carrier frequency;
a modulator receiving the at least one carrier wave and a modulation wave to modulate the at least one carrier wave for application to the at least one electrode;
an electrical modulation generator generating the modulation wave having a predetermined periodicity providing a first period of stimulation and a second period of a different or no stimulation, the predetermined periodicity selected to increase wall movement over continuous stimulation of the glymphatic system or meningeal system by the carrier frequency;
a sensing device measuring cerebral spinal fluid (CSF)/interstitial fluid (ISF) flow in the brain of the human patient or a representative measure of CSF/ISF flow; and
a controller configured to adjust at least one of the at least one carrier wave and the modulation wave to increase CSF/ISF flow in response to a measurement of the sensing device.

2. The device of claim 1 wherein the controller is configured to adjust the predetermined periodicity of the modulation wave in response to a measurement of the sensing device.

3. The device of claim 1 wherein the controller is configured to adjust the first carrier frequency in response to a measurement of the sensing device.

4. The device of claim 1 wherein the controller is configured to adjust the first carrier amplitude in response to a measurement of the sensing device.

5. The device of claim 1 wherein the measurement of the sensing device is a measure of an electrical activity of the brain.

6. The device of claim 5 wherein the sensing device is at least on of an electroencephalogram (EEG) machine and a magnetoencephalography (MEG) machine.

7. The device of claim 1 wherein the measurement of the sensing device is a measure of blood perfusion in the brain.

8. The device of claim 7 wherein the sensing device is at least one of a computerized topography (CT) scanner, magnetic resonance imaging (MRI) scanner, functional magnetic resonance imaging (fMRI) scanner, positron emission tomography (PET) scanner, transcranial ultrasound, and single-photon emission computed tomography (SPECT) scanner.

9. The device of claim 1 wherein the sensing device is at least one of a pupilometer measuring a dilation of the pupil, functional near-infrared spectroscopy (fNIRS) measuring changes in hemoglobin in cerebral blood, a device measuring blood perfusion in the skin of the face or head, and a device measuring an evoked neural signal in the facial or trigeminal nerves.

10. The device of claim 1 wherein at least one electrode is adapted to stimulate at least one of a trigeminal nerve, buccal branch nerve, mental branch nerve, facial branch nerve, vagus branch nerve (e.g., auricular vagus nerve), cervical nerve, sympathetic trunk/sympathetic ganglia, and sympathetic efferent branches.

11. The device of claim 1 wherein the at last one carrier wave comprises first and second carrier waves having first and second distinct frequencies and delivered simultaneously to the at least one electrode.

12. The device of claim 1 further comprising sensors detecting salivary biomarkers indicating a change to CSF flow selected from at least one of amyloid beta peptide, tau protein, lactoferrin, alpha-synuclein, DJ-1 protein, chromogranin A, huntingtin protein, DNA methylation disruptions, and micro-RNA.

13. A method of modifying the function of a glymphatic system or meningeal lymphatic system of a patient comprising:
positioning at least one electrode in close proximity to a nerve of the patient;
generating a carrier wave having a carrier frequency;
generating a modulation wave having a predetermined periodicity providing a first period of stimulation and a second period of no stimulation, the predetermined periodicity selected to increase fluid flow over continuous stimulation of the glymphatic system or meningeal system;
modulating the carrier wave and applying the carrier wave to the electrode;
measuring a cerebral spinal fluid (CSF)/interstitial fluid (ISF) flow in the brain of the patient or a representative measure of CSF/ISF flow; and
adjusting at least one of the carrier frequency and the modulation wave to increase CSF/ISF flow in response to a measurement of the CSF/ISF flow in the brain of the patient or the representative measure of CSF/ISF flow.

14. The method of claim 13 further comprising generating a second carrier wave having a second carrier frequency wherein the second carrier frequency is less than the first carrier frequency and is delivered simultaneously with the first carrier wave.

15. The method of claim 13 further comprising adjusting the second carrier frequency to increase CSF/ISF flow in response to the measurement of the CSF/ISF flow in the brain of the patient.

16. The method of claim 13 further comprising measuring an electrical activity of the brain.

17. The method of claim 13 further comprising measuring a blood perfusion in the brain.

18. The method of claim 13 further comprising adjusting a position of the at least one electrode in response to the measurement of the CSF/ISF flow in the brain of the patient.

19. The method of claim 13 further comprising adjusting a delivery time of the at least one carrier frequency in response to the measurement of the CSF/ISF flow in the brain of the patient and according to a measure of electrical activity of the brain.

20. The method of claim 13 wherein the at least one electrode is positioned over at least one of an inferior alveolar nerve and a mental branch nerve.
